(19) United States
(12) Patent Application Publication
(10) Pub. No.: US 2001/0056235 A1
(43) Pub. Date: Dec. 27, 2001

(54) Medical diagnostic ultrasound instrument with authorization mechanism

(76) Inventors: Jens U. Quistgaard, Seattle, WA (US); Leo R. Catallo, Mercer Island, WA (US)

Correspondence Address: TOWNSEND AND TOWNSEND AND CREW, TWO EMBARCADERO CENTER, EIGHTH FLOOR, SAN FRANCISCO, CA, 94111-3834, US

(21) Appl. No.: 09/811,341

(22) Filed: Mar. 16, 2001

Related U.S. Application Data
(60) Provisional application No. 60/190,224, filed on Mar. 17, 2000.

(51) Int. Cl. A61B 8/00

(52) U.S. Cl. 600/437; 600/441

(57) ABSTRACT

A software security mechanism which restricts modification of software in a programmable diagnostic ultrasound instrument.

### CROSS-REFERENCES TO RELATED APPLICATIONS

This application claims priority from the U.S. Provisional Application No. 60/190,224 filed Mar. 17, 2000 and is related to copending U.S. application Ser. No. 09/564,601 filed May 3, 2000, which are incorporated herein by reference for all purposes.

### BACKGROUND OF THE INVENTION

This invention relates to medical ultrasonic diagnostic systems and, in particular, to ultrasonic diagnostic instruments which employ substantial software or programmable data in their operation.

As is well known, modem ultrasonic diagnostic systems are complex systems, many of which employ computer control circuits. These computer circuits, in turn, are controlled by software and data which is typically stored in some form of addressable memory associated with the instrument. This memory may be, for example, semiconductor memory or a rotating magnetic disk. As ultrasound systems have become more sophisticated, they have employed a greater quantity of software and data in the control of the instrument.

An advantage of extensive use of software programmable instruments that may rely on other stored data for operation is that new capabilities can be added or enabled by modifying this software or data. Also, design or implementation defects in software or data can be corrected by modifying the software or data. Modification of the software or data may be accomplished by introducing new software or data via a portable representation, such as a removable magnetic disk, optical memory, or semiconductor memory, or via an electronic communications mechanism such as a wired or wireless communications network. The same mechanisms may be applied to alter existing software or data as opposed to introducing new software or data.

A significant limitation of current mechanisms for update or modification of data or software associated with diagnostic ultrasound systems is that of control of the update or modification process. Regulatory requirements typically stipulate that manufacturers must keep accurate records of any modification to diagnostic systems, including software or data updates. Thus the modification process must be restricted in some way so that appropriate data can be maintained. In addition, it is desirable to limit access to certain functions of an instrument in order to provide lower cost, limited functionality systems that may later be upgraded with more extensive functionality. Typical mechanisms employed currently to implement such control over the modification process are either cumbersome or ineffective. Service personnel may be sent to a customer site to perform the process, or instruments may be returned to a central facility for update. Either mechanism incurs significant expense in both money and time. Other mechanisms may be employed to allow users of the instrument to perform the update process, but this limits the control that the manufacturer has over the process.

### BRIEF SUMMARY OF THE INVENTION

In accordance with the principles of the present invention, a diagnostic ultrasound instrument is provided with a software security mechanism that effectively restricts modification or replacement of software or data associated with the instrument. Updates to software or data for a particular type of instrument can be developed and easily distributed, but control over the actual update or modification of any specific instrument is retained.

In the preferred embodiment, a “keycode” is generated via an algorithm that takes a unique system identifier and information regarding the modification or update to be performed as inputs. Software in the instrument to be updated prevents any update or modification of the instrument's software or data unless the correct keycode is provided by the person or agency performing the update process. Requiring the person or agency performing the update to obtain the keycode from one or more authorized agencies allows the manufacturer to control such upgrade processes to satisfy both regulatory and feature-control requirements. This mechanism does not require service personnel to perform the update process, nor is any movement of the instrument to an update facility required. Update software, data, and keycodes may be provided via a variety of mechanisms including portable memories, communication networks, facsimile, or voice and manual input via the instrument's user interface.

### BRIEF DESCRIPTION OF THE DRAWINGS

FIG. 1 illustrates in block diagram for the architecture of a typical ultrasound system including control paths from a computer controller.

FIG. 2 illustrates the basic mechanism involved in generating authorization keycodes.

FIG. 3 illustrates the basic mechanism involved in verifying authorization keycodes by the ultrasound instrument.

FIG. 4 illustrates a process for updating software or data in an ultrasound instrument utilizing keycodes.

FIG. 5 illustrates a process for enabling capabilities in an ultrasound instrument utilizing keycodes.

### DETAILED DESCRIPTION OF ILLUSTRATIVE EMBODIMENTS

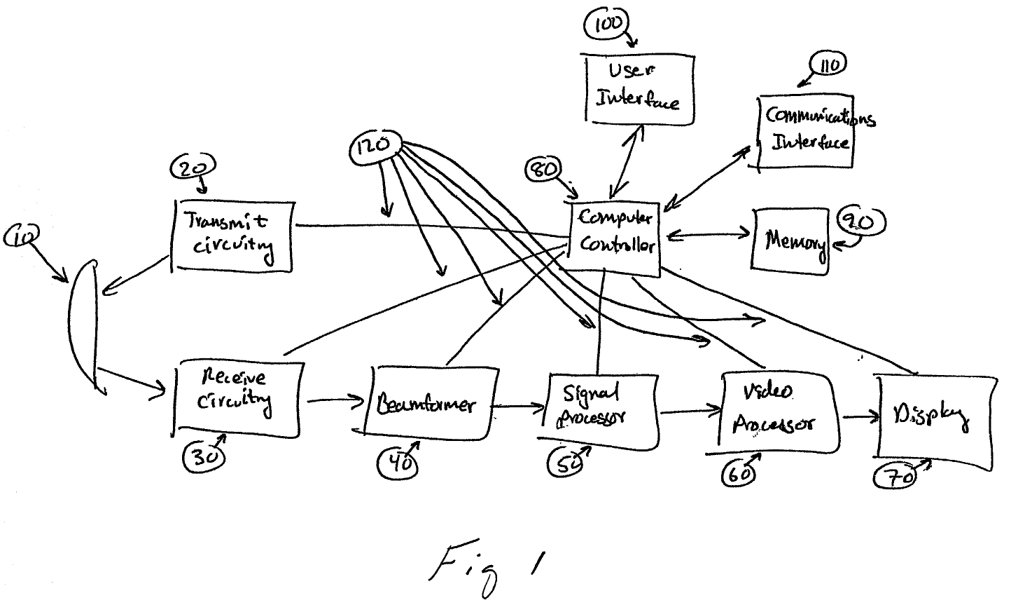

Referring first to FIG. 1; the architecture of a typical ultrasound system to which the present invention may be applied is shown. A transducer array 10 is operatively coupled to transmit subsystem 20 and receive subsystem 30. The transmit subsystem causes electrical signals of appropriate timing, magnitude, and duration to be applied to elements of the transducer array, which causes acoustic waves to emanate from the transducer. Returning echoes are routed to the receive subsystem where they may be amplified and/or filtered.

Amplified and/or filtered signals from the receive subsystem are routed to the beamformer 40 which combines signals into a composite representation via appropriate delay, summation, filtering, and/or other operations. The composite representation is routed to the signal processing subsystem 50, which provides filtering for B-mode signals, and may also provide advanced features such as synthetic aperture mormation, frequency compounding, Doppler processing, speckle reduction, and three-dimensional image formation.

The B-mode and Doppler information is then coupled to the video processor 60 for scan conversion and the production of video output signals. The video output signals may be in digital or analog forms, and are coupled to the display 70. The display may be incorporate a cathode-ray tube or a flat panel display such as a liquid-crystal display.

The computer controller 80 is coupled to controllable subsystems 20, 30, 40, 50 and 60 via control paths 120. In the preferred embodiment, these control paths are combined into one or more buses rather than being implemented separately. The computer controller itself may be a single processor, or may be implemented as a distributed processor incorporating several separate processors. The computer controller is coupled to memory 90 which may consist of a combination of semiconductor memory and other memory mechanisms such as magnetic disks and removable media. Software and data used in control of the ultrasound system is contained in this memory. The computer controller is also coupled to the user-interface subsystem 100 which provides operator control and feedback mechanisms, and to the communication interface 110 which may be coupled to external computers or other communications equipment or infrastructure such as telephone or data communication networks.

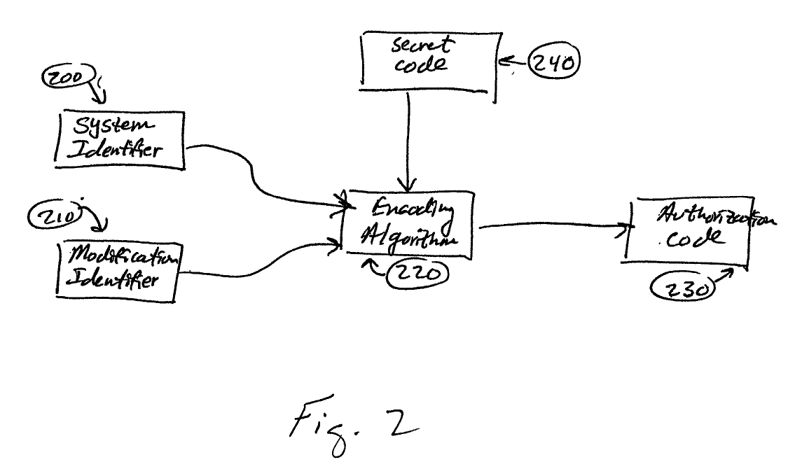

FIG. 2 illustrates the basic mechanism involved in generating authorization keycodes. A system identifier 200, which in the preferred embodiment is the serial number of the ultrasound system or a major component thereof, is supplied as an input to the encoding algorithm 220. An update code 210 representing the update or modification to be performed, which in the preferred embodiment is an alphanumeric code uniquely identifying the operation, is also supplied as an input to the encoding algorithm. A secret code 240, which in the preferred implementation is an alphanumeric code, is also supplied as input to the encoding algorithm.

The encoding algorithm produces an authorization code 230 using these inputs via a mechanism that is difficult to duplicate without access to the detailed implementation of the algorithm and all of its inputs. In the preferred embodiment, this algorithm is implemented as a “one-way function,” the concept of which is well known in the software and cryptography communities.

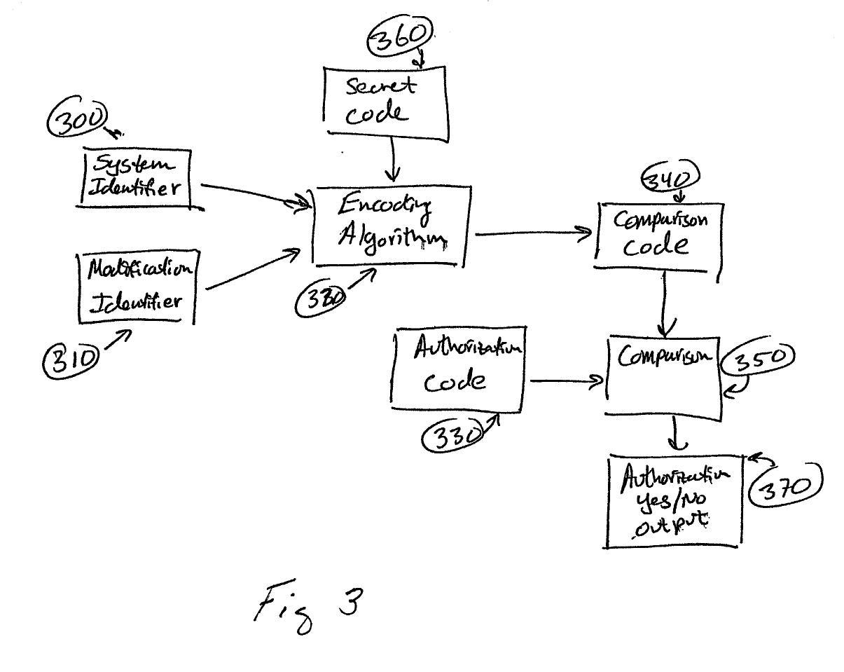

FIG. 3 illustrates the basic mechanism involved in verifying authorization keycodes by the ultrasound instrument. A system identifier 300, which in the preferred embodiment is the serial number of the ultrasound system or a major component thereof, is supplied as an input to the encoding algorithm 320. An update code 310 representing the update or modification to be performed, which in the preferred embodiment is an alphanumeric code uniquely identifying the operation, is also supplied as an input to the encoding algorithm. A secret code 360, which in the preferred implementation is an alphanumeric code, is also supplied as input to the encoding algorithm.

The encoding algorithm produces an comparison code 340 using these inputs via a mechanism identical to that used to generate keycodes as discussed above. Thus if the same inputs are provided to this encoding algorithm, a comparison code identical to a keycode generated as described above is produced. This comparison code and an authorization keycode 330 supplied by the person or agency attempting to perform and update process are supplied as inputs to the comparator 350.

If the comparison code and the authorization keycode are identical, the comparator provides an authorization output 370 which indicates that the correct authorization keycode has been supplied. This output may then be used by other system software or hardware to enable the update process to proceed. If the comparison code and the supplied authorization keycode are not identical, the update process is not allowed to proceed.

In the preferred embodiment, the system identifier and secret code provided as inputs to the encoding algorithm in the verification process illustrated in FIG. 3 are embedded in the ultrasound system and are not changeable by the user. The secret code used in the encoding process should, in the preferred embodiment, not be available for examination by users. The system identifier and secret code used in the verification process should be identical to those used in the keycode generation process for a specific instrument as illustrated in FIG. 2.

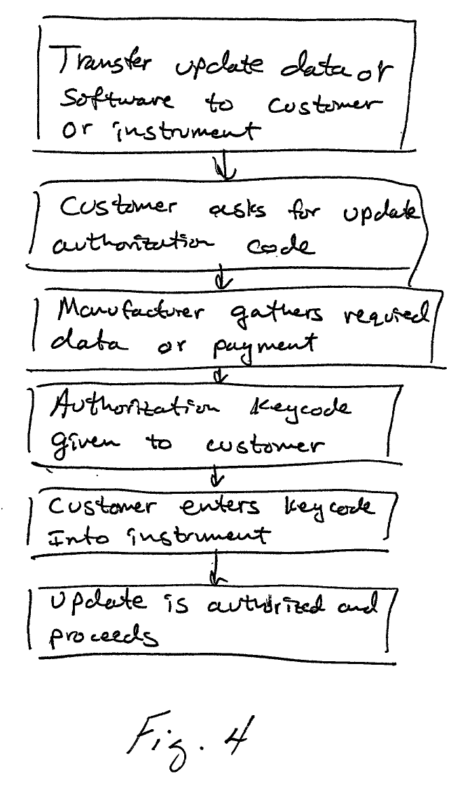

In FIG. 4 we illustrate a typical process for update or replacement of software or data associated with an ultrasonic instruments in accordance with this invention. A data package is provided to the customer by the manufacturer which contains, ideally in machine-readable form, the software or data to be updated in the instrument and an associated update code identifying the update or modification to be performed. This data package may be provided in a portable data representation or via a communication mechanism as discussed previously. The customer then contacts the manufacturer and supplies the system identifier (300 in FIG. 3) in order to obtain an authorization keycode. At the time of this transaction, the manufacturer may gather any information required by regulatory or other agencies. Note that this entire transaction or parts thereof can be automated via a web-site or similar means.

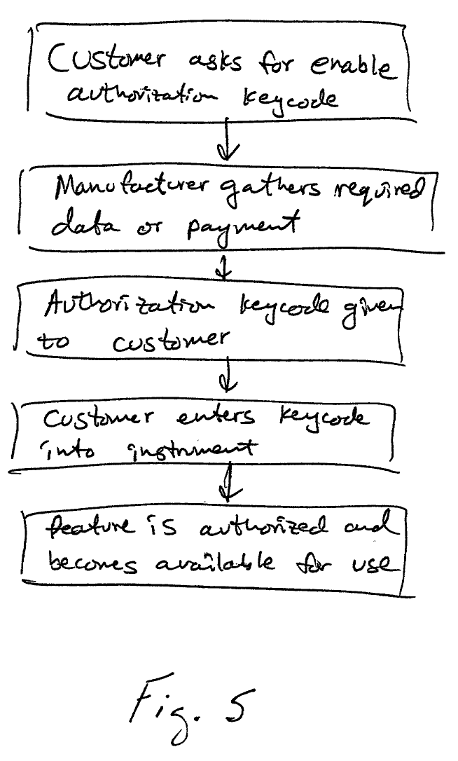

In FIG. 5 we illustrate a typical process for enabling optional features on an ultrasonic instrument in accordance with this invention. A customer or other agent who wishes to enable a feature on an instrument. For illustrative purposes, we assume that the instrument was originally provided with Doppler imaging capability, but that this capability was disabled at the factory. The customer contacts the manufacturer or authorized agent to obtain an authorization keycode for the feature. At the time of this transaction, the manufacturer may gather any information required by regulatory or other agencies, in addition to any payment that may be required. The manufacturer or authorized agent then generates an authorization keycode for the feature (as in FIG. 2) which is provided to the customer. The customer enters this keycode into the ultrasound instrument, where it is validated (as in FIG. 3). Once validated, the instrument allows the new feature (e.g. Doppler imaging) to become active and usable by the customer. Again, note that this entire transaction or parts thereof can be automated via a web-site or similar means.

What is claimed is:

1. In a programmable diagnostic ultrasound instrument having stored software and data for operation control, a software security mechanism which restricts modification of software or data including an algorithm which generates a keycode based on a unique system identifier which allows a person or agency to perform a system or data update.

2. The programmable diagnostic ultrasound instrument of claim 1, wherein the system or data update is performed through a detachable scan head.

3. The programmable diagnostic ultrasound instrument of claim 1, wherein the software security mechanism is a signature generator.

4. An ultrasound instrument having a software library and data for operational control stored on a persistent memory device, and having a means for securely enabling and disabling applications within the software library.
